(19) United States
(12) Patent Application Publication
(10) Pub. No.: US 2009/0286305 A1
(43) Pub. Date: Nov. 19, 2009

(54) Method for non-destructive macromolecule extraction from biological samples on slide

(76) Inventor: Wei-Sing Chu, Silver Spring, MD (US)

Correspondence Address: Nianxiang Zou, 40 Case Street, Gaithersburg, MD, 20878, US

(21) Appl. No.: 12/512,214

(22) Filed: Jul. 30, 2009

Related U.S. Application Data
(63) Continuation-in-part of application No. 11/106,511, filed on Apr. 15, 2005, now Pat. No. 7,588,890.

(51) Int. Cl. C12N 1/08; C07K 1/00

(52) U.S. Cl. 435/270; 435/272

(57) ABSTRACT

A thin layer of a biological sample, such as a section of frozen or preserved tissue sample, a section of fresh or preserved cells, and a mono layer of prokaryotic and eukaryotic cells, is placed on a flat surface of a solid supporting base. Macromolecules, such as DNA, RNA, and proteins, are extracted directly from the thin layer of the biological sample that is attached to the supporting base.

### RELATED APPLICATION

This application is a continuation in part of U.S. patent application Ser. No. 11/106,511, filed Apr. 15, 2005, which is hereby incorporated by reference as if set forth fully herein.

### FIELD OF THE INVENTION

The present invention relates to a method for extracting biological molecules, preferably proteins, DNA, and/or RNA, from biological samples, including, but not limited to, sections of frozen or paraffin-embedded fixed tissues, thin layers of homogenized tissues, tissue cultures, and monolayers or structures close to monolayers of eukaryotic and prokaryotic cells. The morphology of cells after extraction of biological molecules can be maintained without destruction.

### BACKGROUND OF THE INVENTION

With the advent of personalized medicine, comprehensive molecular analysis of human tissue specimens is rapidly becoming a requisite standard for high-throughput exploration of the molecular basis of diseases and for personalized cancer diagnosis and prognosis. Formalin fixation followed by paraffin embedding (FFPE) has been a standard procedure used in over 90% of clinical tissue specimen preparations because of its superior preservation of morphological details, high consistency, ease of processing and handling, and reasonable cost. Formalin preserves tissue morphology by cross-linking proteins and nucleic acids rendering them insoluble under physiological conditions. Due to the widespread use of FFPE tissues, any application of molecular biology technologies should be in compliance with FFPE tissue specimens in order to gain wide clinical acceptance. However, current clinical molecular assays are severely hampered for FFPE tissue specimens because of cross-linked biomolecules. The first problem encountered when applying proteomic and genomic study to FFPE tissues is how to convert cross-linked proteins and nucleic acids from a fixed state into a soluble form while in buffer solutions.

The finding of antigen retrieval by heating FFPE tissues in water or buffer solution has shed light on molecule extraction from FFPE tissues. In 1990s, several groups reported that digesting de-paraffinized FFPE tissues with proteinase K successfully released DNA and RNA for PCR amplification although mRNA size was greatly reduced. Later, protein extraction methods were formulated essentially by heating de-paraffinized FFPE tissues in RIPA buffer. Researchers are still working to fine-tune protein extraction methods for proteomic studies. So far, all current tissue extraction methods require chopping or slicing tissue samples into small pieces, a process called homogenization. Pathological labs routinely distribute tissue samples in the form of sections on microscope slides. To extract biomolecules, researchers must scrape the tissue sections off the slides into extraction tubes. This process is time-consuming and may lead to unwanted sample loss and contamination. At the same time, tissue morphology is destroyed.

DNA and RNA extraction from formalin fixed and paraffin-embedded (FFPE) tissue samples frequently require several hours to overnight incubation for proteinase K digestion. Protein extraction from FFPE tissue samples takes about 2 hours under various temperatures and in certain buffer solutions. For instance, the original protein extraction protocol of Ikeda et al. was to cut a tissue sample into 3- to 10-micron-thick tissue sections incubated in microcentrifuge tubes in RIPA buffer at 100 .degree.C for 20 minutes, then at 60 .degree.C for 2 hours. The existing protocols for molecule extraction from FFPE tissues all have at least these three limitations: 1) they require long processing times, especially for nucleic acid extraction; 2) they call for tissue homogenization or scraping tissue sections off slides; 3) the tissue morphology is destroyed after extraction. The present invention is about a nondestructive molecule extraction (NDME) technique that extracts biological molecules from tissue sections or monolayer cells that are attached to microscopic slides.

### SUMMARY OF THE INVENTION

The present invention provides a method to extract biological molecules from biological samples directly on-slides, without destructing the cell or tissue morphologies. NDME also makes it possible for molecular fractionation to be performed to cells in tissue sections and cytology smears.

NDME method abolishes the need of homogenizing tissue samples. By performing extraction on slide, NDME can be easily adapted in the routine pathology laboratories streamlining molecular and histological analyses. It can be applied in a multifunctional extraction/incubation system that can be used for on-slide extraction of proteins and nucleic acids, antigen retrieval, pretreatment, as well as histopathology assays such as immunohistochemistry (IHC) staining and in situ hybridization (ISH).

In one embodiment, a thin layer of a biological sample, e.g., cells in monolayer or close to monolayer, a section of a fixed and processed tissue sample, or a section of a snap-frozen tissue sample, is placed on a surface of a microscopic slide. A small volume of extraction solution, usually in the range of 5-250 .mu.L, depending on size of the sample, is placed over the sample on slide. It is then incubated for a certain amount of time at a certain temperature to reach required level of extraction of biomolecules from the biological sample. After incubation, the extraction buffer, containing biomolecules extracted from the biological sample, is collected for downstream assays.

In one embodiment, the extraction solution over the thin layer of biological sample is covered by an inert liquid, such as mineral oil, to prevent evaporation during incubation.

In another embodiment, extraction solution over the thin layer of biological sample on slide is covered by a surface of a solid object that forms a capillary space over the thin layer of biological sample on flat surface of the supporting base to prevent evaporation. The solid object can be, but is not limited to, a cover slip, a slide chamber, or a stand where the supporting base can rest upon in a face-down position. In one embodiment, extraction solution is added before the capillary space is formed. In another embodiment, extraction solution is added after the capillary space is formed.

In one embodiment of the invention, microscopic observation is performed to the tissue sections on slide to identify areas of interest on tissue sections. Unwanted areas of tissue sections can be removed before extraction begins. In another embodiment, since unextracted molecules are still left on slides, one can do on-slide assays, such as IHC and ISH, on the same slides after extraction is completed.

In another embodiment, the supporting base of the tissue section is a transparent thin sheet made of glass or synthetic polymers, which has a certain level of strength and rigidity, such as a cover slip, or a plastic film similar to an X-ray film, and which can be easily cut by cutters. The area of tissue section of interest together with attached supporting base can be cut out and placed in a container holding a small volume of extraction buffer. Incubation for extraction of biomolecules can be performed in the container. In this case, unextracted biomolecules are still attached to a small piece of supporting base. On-slide assays, such as IHC and ISH, can be performed on the small piece of supporting base.

When incubation is performed at a relatively high temperature, e.g., higher than 50 .degree.C, or when relatively small volumes of extraction buffer are used, e.g., less than 100 .mu.L, or for relative long period of time, e.g., longer than 20 minutes, incubation may need to be performed in a humid environment. Humid environments can be constructed with a water bath, a humidity incubator, a steamer, or by introducing humid air or water vapor into the container that holds the extracted sample.

### BRIEF DESCRIPTION OF THE FIGURES

FIG. 1. Working hypothesis of the NDME technology.

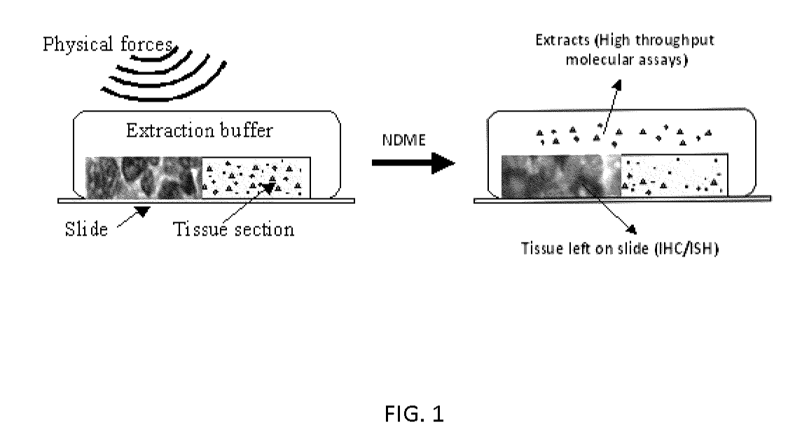

FIG. 2. Schematic diagram of a NDME snap-on slide chamber.

FIG. 3. A time course study of extracted proteins and tissue morphology after NDME treatment for 5, 10, 15, 20, and 30 minutes, respectively. A) IHC staining of tissue sections with anti-CD5 antibody. B) SDS-PAGE analysis of proteins extracted from tissue sections in Panel A.

FIG. 4. A) RNA extracted by NDME from 6 cases (2-7, #1=water control) of 30-year FFPE retinal sections, generating RT-PCR amplicons of 367 bp from beta-actin gene. M=100 bp DNA ladder. B) RNA-ISH of consecutive sections of lymph node with infectious mononucleosis. Blue signals show Epstein-Barr virus early RNA (EBER) hybridization. C) PCR of NDME extracts from FFPE and frozen tissue sections generated DNA of up to 1,309 bp. D) CISH detection of the c-Myc translocation in Burkitt's lymphoma tissue sections with/without NDME. Inset showed translocation in one cell. The c-Myc translocation was obvious in Burkitt's lymphoma tissue sections before and after NDME.

FIG. 5. NDME extraction of laser micro dissected prostate tissue sections, microscopic view. Total proteins and AMACR could be detected in NDME extract. A, B, C, D, E indicated 5 specimens.

FIG. 6. Western blots and IHC for different type of proteins after NDME. NDME was performed on the following tissue specimens: LN=reactive lymph node; HIV+=HIV+AIDS lymph node; ALCL=anaplastic large cell lymphoma; BL=Burkitt's lymphoma. A and B): NDME extracts analyzed by Western blots as detected by anti-CD20, anti-HIV p24, anti-CD30, and anti-cyclin E antibodies. C): post-NDME IHC of the ALCL (upper) and BL sections (lower) stained by anti CD30 (left) and anti-cyclin E (right).

FIG. 7. A) Western Blots for HER2 protein extracted by a 30-min NDME. Lanes 1 and 2 each represents the HER2 protein from a breast cancer tissue section with a score of 3+ and from the cell line T47D serving as a positive control. B) IHC staining for HER2 protein on the same section after NDME and another section treated by routine AR.

### DETAILED DESCRIPTION OF THE INVENTION

In theory, extraction of a specific biomolecule type from tissue sample is a process of reaching equilibrium between the molecules being released into extraction buffer and those remaining in the tissue sample. Under the same physical condition, the milder the extraction buffer, the fewer biomolecules are extracted. On the other hand, using the same extraction buffer, the harsher the extraction condition, the more molecules are extracted.

Extraction solution is designed to remove macromolecules of interest from the samples to be extracted, and, at the same time maintain a certain degree of integrity of the removed macromolecules of interest for downstream liquid-based assays. The term “a certain degree of integrity” here implies that micromolecules of interest extracted by NDME can be assayed in the downstream liquid-based analysis to generate data reflecting the existence of the macromolecules of interest in the samples being extracted. While in the conventional antigen retrieval or pretreatment procedures in on-slide assays, such as IHC and ISH, the antigen retrieval or pretreatment buffers do not, or are not designed to, remove macromolecules of interest from the samples to be analyzed, and, after incubation, antigen retrieval or pretreatment buffers are not collected for downstream liquid-based assays, such as polymerase chain reaction (PCR), gel electrophoresis, ELISA, Western Blot, Northern Blot, mass spectrometry, and many others.

Besides tissue samples, NDME can be used to extract other cell-containing samples. The term “a thin layer of biological sample” refers to a tissue section, a cytological smear, or suspended cells being laid out in a monoclayer or close to a monoclayer, therefore, it refers to a layer of biological sample that is less than 20 micron in thickness. Tissue sections are usually cut to 2-10 micron in thickness, reflecting about one quarter to one whole mammalian cell in diameter. Suspended cells include, but not limited to, cells from body fluids, cells separated from tissues, and cultured cells of both eukaryotic and prokaryotic nature.

In the NDME procedure, biomolecules are released controllably from one surface of the tissue section into the extraction buffer while the other surface is protected by attachment to the support. It has been demonstrated that NDME extracted high quantity and wide spectrum of biomolecules suitable for various downstream molecular analyses from formalin-fixed and paraffin-embedded (FFPE) tissue sections. FFPE tissue samples are among the most difficult tissue samples to be extracted. The extraction efficiency of NDME can be monitored by observing tissue morphology left on slide. The tissue sections after controlled partial NDME can be used for histological, immunostaining, and in situ hybridization analyses.

The simplest embodiment of the invention is laying extraction buffer on top of a tissue section or a monolayer of cells that are attached to a microscopic glass slide, covering it with an inert liquid or a chamber slide to prevent evaporation of the extraction buffer and condensation of moisture into extraction buffer during incubation. The microscopic glass slide can be replaced with any solid supporting base with a flat surface that is resistant to erosion of extraction solutions and high temperatures. Since the volume of extraction solution used in NDME is small, for prolonged incubation or incubation at a high temperature, it is required that incubation is performed in a humid environment, such as in a water bath or humidified incubation chamber.

In case of extraction of biomolecules from formalin-fixed and paraffin-embedded tissue samples, incubation at temperatures close to 100 .degree.C is generally required. Incubation at 100 .degree.C or over can be achieved by infusion of water steam into an incubation chamber. When paraffin-embedded tissue sections are extracted by NDME, deparaffinization must be performed, typically by a 2-minute immersion in xylene for 5 rounds, 100% alcohol twice, 95% alcohol once to rehydrate and then air-drying at room temperate for 5 minutes.

A slide chamber functions to form a capillary space over the thin layer of the biological sample. In addition to preventing evaporation and condensation, the slide chamber also helps to spread the extraction solution over the thin layer of biological sample by the capillary force. Other solid objects that can form a capillary space with the sample surface of the glass slide can be used in place of the slide chamber. In one embodiment, a microscopic glass slide can be placed with the sample face down on a supporting object which has a surface to form a capillary space with the sample face of the microscopic glass slide.

The sample surface of the slide chamber or other solid objects that are used to form the capillary space can change from rectangular to circular or elliptical. Rectangle-shaped chambers hold larger buffer volume and cover more surface area, suitable for large sample areas (large tissue sections), while circular chambers are better suitable for small sample areas (small tissue sections). The sample surface of the slide chamber or other solid objects can be changed from flat to slightly convex or concave at the center. There may be an opening(s) in the slide chamber or other solid objects for one to add and retrieve extraction solution easily to and from the capillary space between sample surface of slide chamber or other solid objects and sample surface of the glass slide or the supporting base in other forms.

The advantage for having a convex at the center of the sample surface of a slide chamber or other solid objects is that when extraction buffer is added through a central hole, it will stay around the center area where the tissue section is normally located by the capillary force. When more buffer is added, it will spread to the more spacious peripheral space by expansion at higher temperatures. After cooling, the buffer will return to the center by the capillary force to facilitate retrieval. Elliptical shaped slide chambers with adding/retrieving hole(s) at center or on the ends will also be proper. The NDME procedure is very dynamic and allows for modifications in light of different situations.

In many situations, one must be able to analyze specific cell populations within the context of their heterogeneous tissue microecology. Therefore, a tissue section often needs to be dissected to remove unwanted areas of tissue or cells in the section before subjecting to extraction. Such dissection is generally done under a microscope. Laser-capture microdissection (LCM) is a method to procure subpopulations of tissue cells under direct microscopic visualization. LCM technology can harvest the cells of interest directly or can isolate specific cells by cutting away unwanted cells to give histologically pure enriched cell populations. The NDME method makes it easier to perform such dissections. Instead of capturing the area(s) of interest from the tissue section by a laser technology, one can simply remove the unwanted area(s) from the tissue section by scraping it off the slide with a razor, a knife, a scalpel, or other hard object with a sharp edge.

Besides glass slides, it is also possible that the supporting base holding the thin layer of biological sample is a thin sheet of glass, e.g. a cover slip, or a film made of transparent synthetic polymers. In both cases, the supporting base can be conveniently cut by a cutter, e.g., a diamond cutter to cut a glass cover slide or a pair of surgical scissors to cut the film of synthetic polymer. In addition to the option of scraping unwanted area(s) of tissue section off the supporting base, the wanted area(s) of tissue section with the underlining supporting base can be cut out. In this case, NDME is performed in extraction tubes on a tiny piece of supporting base with the wanted area of tissue section attached to it. What is remained on the tiny piece of supporting base after extraction can still be analyzed by morphological examinations such as H&E, IHC, or ISH stains.

Physical forces, such as ultrasound, can be used in NDME procedures in expediting extraction of biomolecules. Low frequency ultrasound has been widely used in tissue homogenization. It has been demonstrated that high frequency ultrasound can promote reagent penetration of tissue cells in fixation process. It is sound to believe that both low and high frequency ultrasounds will help in NDME especially for extensive extraction on tough over-fixed FFPE specimens. In cases where low stringency extraction buffers are needed, e.g., for better compatibility with downstream molecular assays, ultrasound agitation may be highly desirable to increase extraction efficiency. Ultrasound can also increase efficiency of IHC and ISH processes. Ultrasounds of various parameters (Table 1) can be used by the NDME system.

TABLE 1
Ultrasound parameters that can be used in NDME.

| | Condition 1 | | Condition 2 | | Condition 3 | | Condition 4 | | Condition 5 | | Condition 6 | |
|---|---|---|---|---|---|---|---|---|---|---|---|---|
| Frequency | 20-40 | kHz | 20-40 | kHz | 100-200 | kHz | 100-200 | kHz | 0.5-1 | MHz | 0.5-1 | MHz |
| Intensity | 1-5 | w/cm² | 5-20 | w/cm² | 1-5 | w/cm² | 5-20 | w/cm² | 1-5 | w/cm² | 5-20 | w/cm² |

An NDME device can be designed to function for both extraction and binding/hybridization reactions. The uniqueness of an NDME device is its inclusion and accommodation of slides, slide chambers, humidity control, steam pressure control, time control, a wide range temperature control (0 .degree.C-120 .degree.C), and possibly an ultrasound unit. An NDME device will fill the blank of such combined functions in research and clinical communities.

Buffer components, extraction time, temperatures, and methods of tissue preparation all affect the NDME procedure. The ideal buffers and protocols should facilitate controlled release of biomolecules of interest suitable for the downstream molecular analyses. There will be optimized buffers and protocols for various tissue types prepared by different methods. One can establish a mild extraction buffer system addressing both molecule extraction and tissue morphology preservation. One can also develop a harsher buffer system that can extract biomolecules as completely as possible from tissue specimens.

Composition of detergents, salts, and buffer pH in the extraction solution affect efficiency of extraction and morphology of cells remaining on slides. A partial extraction buffer and a complete extraction buffer may need to be developed separately for both proteins and nucleic acids. SDS is the most frequently used detergent in extraction solutions. Other chemical reagents, such as CHAPS, NP-40, and urea, may also affect extraction efficiency. From our preliminary data, the effect of pH on extraction efficiency seems highly dependent on tissue type. Buffers with extremely high or low pH value may not preserve tissue morphology, and buffers with near neutral pH may be better suited for downstream proteomic/genomic analysis.

For the partial extraction, tissue morphology should be well maintained after NDME, otherwise the buffer needs to be modified and retested. To maintain morphological details, the optimized buffers should be able to extract 10%-25% of total molecules of interest from the tissue section. For the complete extraction, incubation can be extended for up to 2 hrs with harsher extraction solution. When harsh extraction solution is used, it is often necessary to further purify or enrich biomolecules of interest for downstream molecular assays since excess salt or detergent concentration often interferes with biological reactions.

NDME works effectively for tissues preserved by various methods, including under- and over-fixed FFPE, snap-frozen, and alcohol-fixed tissues. NDME has been successfully used on archived tissues, which have been stored in tissue blocks for decades. No cross-links exist in frozen and alcohol fixed tissues, therefore, less harsh treatment (i.e. lower temperature, shorter incubation, lower detergent concentration) will be needed for tissues prepared by these methods.

Though the effect might be significantly different across buffers, it is predicted that high temperature produces higher extraction yield due to the known effect of temperature on extraction of biomolecules from preserved samples. Longer extraction time may also generate more soluble molecules. There is an optimal time and temperature range for obtaining both satisfactory extraction and sharp and consistent IHC/ISH signals on slide.

### Example 1

### Snap-on Slide Chamber for NDME

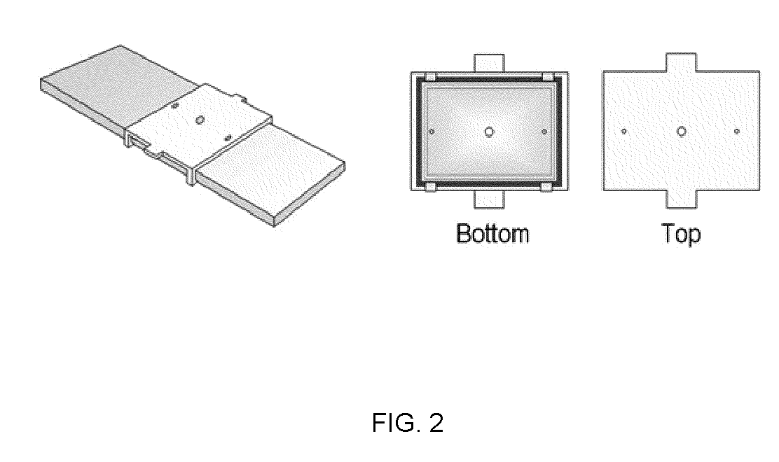

The specially designed snap-on slide chambers are used to spread the extraction buffer over the tissue sections on slide evenly and prevent buffer evaporation and condensation of steam into the extraction buffer (FIG. 2). For consistent and uniform tissue extraction with a small volume (20 to 100 ul) of buffer, the slide chamber design is critical for efficient manipulation of the extraction process.

### Example 2

### Application of NDME in Kinetics Studies on Biomolecule Extraction

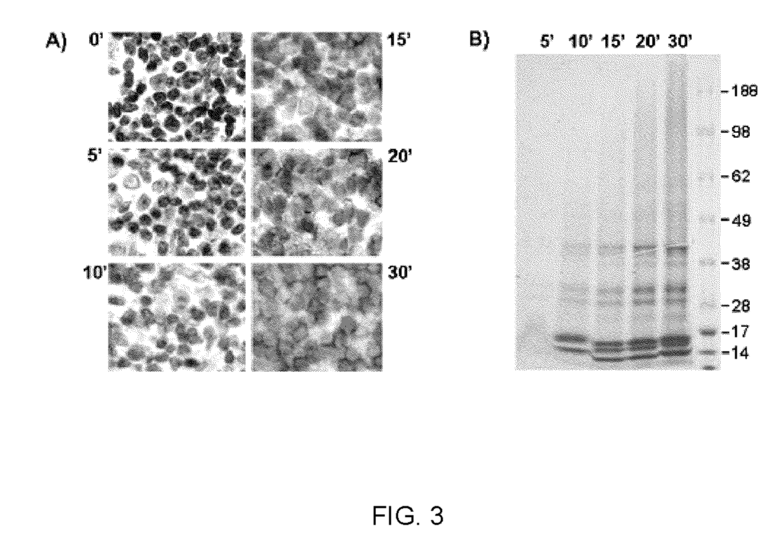

NDME can be used to perform kinetics studies on biomolecule extraction from monolayer cells or tissue sections. It also has potential to be used in protein enrichment or cellular fractionation. To do this, an extraction buffer can be added over the tissue section, incubated for 5 to 10 min and recovered, a second addition of the same amount of the same extraction buffer can be applied for incubation under the same condition and recovered. This procedure can be repeated up to 5 times. The extracts from each step can be analyzed by Western blot and/or dot blot (FIG. 3).

### Example 3

### Application of NDME to Needle Biopsy Samples

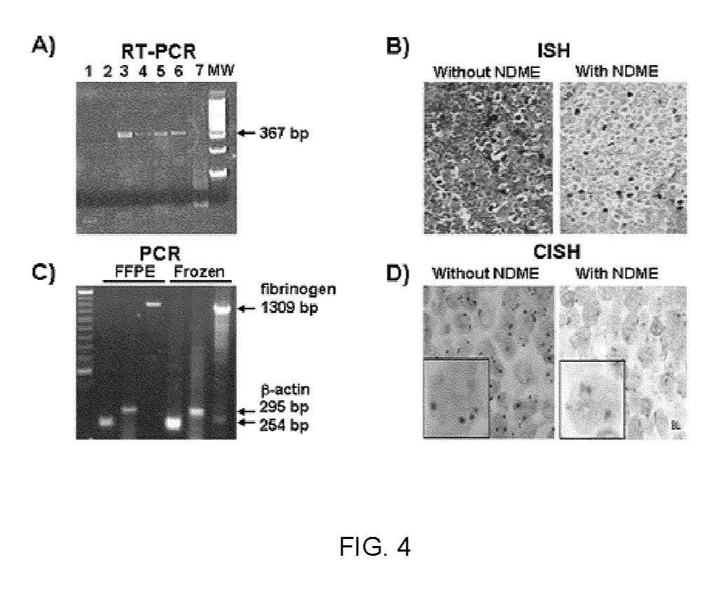

Needle biopsy is currently very common for pathological diagnosis. NDME works for a single slide section of typical needle biopsy samples, as small as 1×1×1 mm3. We extracted enough DNA by NDME from FFPE brain needle biopsy specimens for PCR reactions to study the loss of heterozygosity in chromosomes 1p and 19q for diagnosis and treatment for patients with oligodendroglioma. Furthermore, needle biopsy or laser micro dissected samples can also be extracted by NDME to give informative protein and DNA analysis, as shown in FIG. 4. FFPE prostate tissues after laser micro dissection contain about 6,000 cells.

### Example 4

### Application of NDME to Microdissected Samples

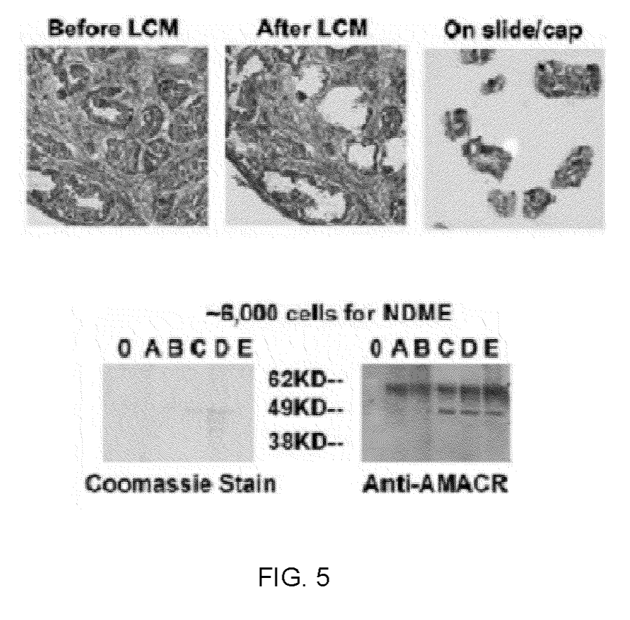

NDME extracts of micro dissected sections from 5 individual cases were analyzed on SDS-PAGE for total protein and Western blot detection of prostate cancer-related AMACR expression. Total proteins as indicated by Coomassie blue staining were of tiny amounts. AMACR signals were obvious in all 5 cases of dissected neoplastic prostate lesions via Western blot (FIG. 5) and IHC (data not shown).

### Example 5

### Western Blot Analysis to Proteins Extracted by NDME

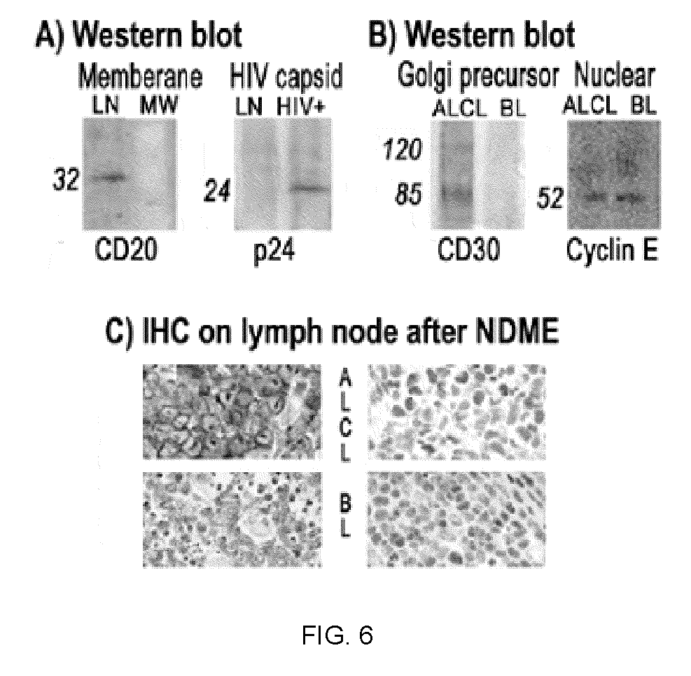

To investigate protein integrity (size) and antigenicity, various proteins extracted from archived FFPE sections were analyzed by Western blot. Proteins of various types can be effectively extracted by NDME for molecular analysis providing information on the size(s) and quantity of proteins, while IHC could be performed after NDME to provide details of cellular morphology and the distribution of protein expression (FIG. 6).

### Example 6

### Controlled Partial NDME

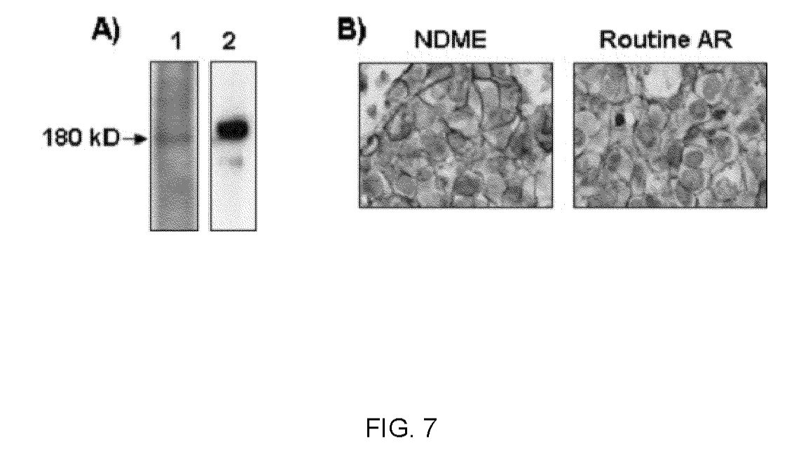

A FFPE breast tissue section with 3+HER2 overexpression was extracted by controlled partial NDME. The extract was subjected to SDS PAGE and Western blot and the treated tissue sections on slides were stained by routine HER2 IHC assay (HercepTest™, DAKO). The 180-kDa HER2 protein could be detected by Western blot (FIG. 7A). The IHC staining result for the tissue section treated with NDME as antigen retrieval was indistinguishable from the one using the routine AR by the standard protocol (FIG. 7B).

While the invention has been disclosed in this patent application by reference to the details of preferred embodiments of the invention, it is to be understood that the disclosure is intended in an illustrative rather than in a limiting sense. As it is contemplated that modifications will readily occur to those skilled in the art, within the spirit of the invention and the scope of the appended claims.

### LIST OF REFERENCES

1. Fox C H, Johnson F B, Whiting J, Roller P P. Formaldehyde fixation. J Histochem Cytochem 1985; 33(8):845-853.
2. Cheng G, Shi Y, Sturla S J, et al. Reactions of formaldehyde plus acetaldehyde with deoxyguanosine and DNA: formation of cyclic deoxyguanosine adducts and formaldehyde cross-links. Chem Res Toxicol. 2003; 16(2):145-52.
3. Metz B, Kersten G F, Hoogerhout P, et al. Identification of formaldehyde-induced modifications in proteins: reactions with model peptides. J Biol Chem. 2004; 279(8):6235-43.
4. Neubauer A, Neubauer B, He M, et al. Analysis of gene amplification in archival tissue by differential polymerase chain reaction. Oncogene 1992; 7: 1019-1025.
5. von Weizsacker F, Labeit S, Koch H K, Oehlert W, Gerok W, Blum H E. A simple and rapid method for the detection of RNA in formalin-fixed, paraffin-embedded tissues by PCR amplification. Biochem. Biophys. Res. Commun. 1991; 174: 176-180.
6. Ikeda K, Monden T, Kanoh T, et al. Extraction and analysis of diagnostically useful proteins from formalinfixed, paraffin-embedded tissue sections. J Histochem Cytochem 1998; 46(3):397-403.
7. Izawa H, Yamamoto H, Ikeda M, et al. Analysis of cyclin D1 and CDK expression in colonic polypscontaining neoplastic foci: a study of proteins extracted from paraffin sections. Oncol Rep 2002; 9(6):1313-1318.
8. Crockett D K, Lin Z, Vaughn C P, Lim M S, Elenitoba-Johnson K S. Identification of proteins from formalinfixed paraffin-embedded cells by LC-MS/MS. Lab Invest. 2005; 85(11): 1405-15.
9. Shi S R, Liu C, Balgley B M, Lee C, Taylor C R. Protein extraction from formalin-fixed, paraffin-embedded tissue sections: quality evaluation by mass spectrometry. J Histochem Cytochem. 2006; 54(6):739-43.
10. Chu W S, Liang Q, Liu J, Wei M, Winters M, Liotta L, Sandberg G, Gong M: “A Nondestructive Molecule Extraction Method Allowing Morphological and Molecular Analyses Using a Single Tissue Section” Lab. Invest. 2005, 85(11):1416-28.
11. Becker K F, Schott C, Hipp S, Metzger V, Porschewski P, Beck R, Nahrig J, Becker I, Hofler H. Quantitative protein analysis from formalin-fixed tissues: implications for translational clinical research and nanoscale molecular diagnosis. J. Pathol. 2007; 211(3):370-8.
12. Huang S N. Immunohistochemical demonstration of hepatitis B core and surface antigens in paraffin sections. Lab Invest. 1975; 33(1):88-95.
13. Hausen P, Dreyer C. Urea reactivates antigens in paraffin sections for immunofluorescent staining. Stain Technol. 1982; 57(5):321-324.
16. Rait V K, O'Leary T J, Mason J T. Modeling formalin fixation and antigen retrieval with bovine pancreatic ribonuclease A: I. structural and functional alterations. Lab Invest 2004, 84:292-299.
17. Rait V K, Xu L, O'Leary T J, Mason J T. Modeling formalin fixation and antigen retrieval with bovine pancreatic ribonuclease A: II. Interrelationship of cross-linking, immunoreactivity, and heat treatment. Lab Invest 2004, 84:300-306.
18. Chu W S, Furusato B, Wong K, Sesterhenn I A, Mostofi F K, Wei M Q, Zhu Z, Abbondanzo S L, Liang Q. Ultrasound-accelerated formalin fixation of tissue improves morphology, antigen and mRNA preservation. Mod Pathol. 2005; 18(6):850-63.
19. Pan L, Diss T C, Peng H, Isaacson P G. Immersion-histo polymerase chain reaction: a practical tool for visualization of single-copy genes in tissue sections. Am J. Pathol. 1997 April; 150(4):1245-52.
20. Espina V, Wulfkuhle J D, Calvert V S, VanMeter A, Zhou W, Coukos G, Geho D H, Petricoin E F 3rd, Liotta L A. Laser-capture microdissection. Nat Protoc. 2006; 1(2):586-603.

What is claimed is:

1. A method for extracting biological molecules from a biological sample, comprising:
a) placing a thin layer of said biological sample onto a flat surface of a supporting base;
b) contacting said thin layer of said biological sample with a small volume of extraction solution, said extraction solution facilitates dissolution of predetermined biological molecules from said biological sample;
c) incubating at a temperature for a length of time; and,
d) collecting said extraction solution which contains said predetermined biological molecules extracted from said biological sample.

2. A method according to claim 1, wherein incubation is performed in a humidity chamber.

3. A method according to claim 1, further comprising covering said small volume of extraction solution over said thin layer of biological sample on said flat surface of said supporting base, to prevent evaporation of said extraction solution and condensation of water vapor into said extraction solution.

4. A method according to claim 1, wherein said biological sample is a paraffin-embedded tissue sample, and wherein a step of de-paraffinization is inserted between step a and step b.

5. A method according to claim 1, wherein the following step is inserted between step a and step b: placing a surface of a solid object close to said thin layer of biological sample to form a capillary space between said surface of said solid object and said flat surface of said supporting base.

6. A method according to claim 1, wherein the following step is inserted between step b and step c: placing a surface of a solid object close to said thin layer of biological sample to form a capillary space between said surface of said solid object and said flat surface of said supporting base.

7. A method according to claim 1, wherein said supporting base is a transparent supporting base that facilitates microscopic examination.

8. A method according to claim 7, further comprising the following two steps inserted between steps a and b: identifying an area of interest on said thin layer of biological sample under a microscope, and, removing unwanted area(s) on said thin layer of biological sample from said transparent supporting base.

9. A method according to claim 8, wherein said unwanted area(s) on said thin layer of biological sample is removed from said supporting base by scraping with an edge of a hard object.

10. A method according to claim 8, wherein said transparent supporting base is a flat sheet made of glass or synthetic polymers, and wherein said unwanted area(s) on said thin layer of biological sample is removed from said supporting base by cutting out said area of interest on said thin layer of biological sample attached to said transparent supporting base.

11. A method according to claim 10, wherein said area of interest on said thin layer of biological sample attached to said transparent supporting base is contacted by said small volume of extraction buffer in a container.

12. A method according to claim 1, further comprising applying ultrasound to said small volume of extraction solution during incubation.

13. A method according to claim 1, further comprising subjecting said small volume of extraction solution collected after incubation to downstream solution-based analysis.

14. A method according to claim 1, further comprising subjecting said thin layer of said biological sample remaining on said flat surface of supporting base to slide-based analysis after incubation and collection of said extraction solution.

15. A method for extracting biological molecules from a thin layer of biological sample attaching to a flat surface of a supporting base, comprising:
a) contacting said thin layer of said biological sample with a small volume of extraction solution, said extraction solution facilitates dissolution of predetermined biological molecules from said biological sample,
b) incubating at a temperature for a length of time, and,
d) collecting said extraction solution which contains said predetermined biological molecules extracted from said biological sample.

16. A method according to claim 15, wherein incubation is performed in a humidity chamber.

17. A method according to claim 15, further comprising covering said small volume of extraction solution over said thin layer of biological sample on said flat surface of supporting base, to prevent evaporation of said extraction solution and condensation of water vapor into said extraction solution.

18. A method according to claim 15, wherein said thin layer of biological sample is a paraffin-embedded tissue section, and wherein a step of de-paraffinization is inserted before step a.

19. A method according to claim 15, wherein the following step is inserted before step a: placing a surface of a solid object close to said thin layer of biological sample to form a capillary space between said surface of said solid object and said flat surface of said supporting base.

20. A method according to claim 15, wherein the following step is inserted between step a and step b: placing a surface of a solid object close to said thin layer of biological sample to form a capillary space between said surface of said solid object and said flat surface of said supporting base.

21. A method according to claim 15, wherein said supporting base is a transparent supporting base that facilitates microscopic examination.

22. A method according to claim 21, further comprising the following two steps inserted before steps a: identifying an area(s) of interest on said thin layer of biological sample under a microscope, and, removing unwanted area(s) on said thin layer of biological sample from said transparent supporting base.

23. A method according to claim 22, wherein said unwanted area(s) on said thin layer of biological sample is removed from said supporting base by scraping with an edge of a hard object.

24. A method according to claim 22, wherein said transparent supporting base is a flat sheet made of glass or synthetic polymers, and wherein said unwanted area(s) on said thin layer of biological sample is removed from said transparent supporting base by cutting out said area(s) of interest on said thin layer of biological sample attaching to said transparent supporting base.

25. A method according to claim 24, wherein said area of interest on said thin layer of biological sample attaching to said transparent supporting base is contacted by said small volume of extraction buffer in a container.

26. A method according to claim 15, further comprising subjecting said small volume of extraction solution collected after incubation to downstream solution-based analysis.

27. A method according to claim 15, further comprising applying ultrasound to said small volume of extraction solution during incubation.

28. A method according to claim 15, further comprising subjecting said thin layer of said biological sample remaining on said flat surface of supporting base to slide-based analysis after incubation and collection of said extraction solution.
